(19) United States
(12) Patent Application Publication
(10) Pub. No.: US 2023/0233568 A1
(43) Pub. Date: Jul. 27, 2023

(54) ANTICANCER COMBINATION THERAPY

(71) Applicant: Boehringer Ingelheim International GmbH, Ingelheim am Rhein (DE)

(72) Inventors: Marco Hans HOFMANN, Vienna (AT); Michael GMACHL, Vienna (AT); Fabio SAVARESE, Maria Enzersdorf (AT)

(21) Appl. No.: 18/002,570

(22) PCT Filed: Jun. 23, 2021
(86) PCT No.: PCT/EP2021/067089

§ 371 Date: Dec. 20, 2022

Related U.S. Application Data
(60) Provisional application No. 63/043,333, filed on Jun. 24, 2020.

(51) Int. Cl. A61K 31/519

(52) U.S. Cl. CPC A61K 31/519

(57) ABSTRACT

The invention describes anti-cancer therapies comprising using a SOS1 inhibitor in combination with a KRAS G12C inhibitor, each as described herein.

### FIELD OF THE INVENTION

The invention describes anti-cancer therapies comprising using a SOS1 inhibitor in combination with a KRAS G12C inhibitor, each as described herein.

### BACKGROUND OF THE INVENTION

Ras family proteins including KRAS (V-Ki-ras2 Kirsten rat sarcoma viral oncogene homolog), NRAS (neuroblastoma RAS viral oncogene homolog) and HRAS (Harvey murine sarcoma virus oncogene) and any mutants thereof are small GTPases that exist in cells in either GTP-bound or GDP-bound states (McCormick et al., J. Mol. Med. (Berl)., 2016, 94(3):253-8; Nimnual et al., Sci. STKE., 2002, 2002(145):pe36). The Ras family proteins have a weak intrinsic GTPase activity and slow nucleotide exchange rates (Hunter et al., Mol. Cancer Res., 2015, 13(9):1325-35). Binding of GTPase activating proteins (GAPs) such as NF1 increases the GTPase activity of Ras family proteins. The binding of guanine nucleotide exchange factors (GEFs) such as SOS1 (Son of Sevenless 1) promote release GDP from Ras family proteins, enabling GTP binding (Chardin et al., Science, 1993, 260(5112):1338-43). When in the GTP-bound state, Ras family proteins are active and engage effector proteins including C-RAF and phosphoinositide 3-kinase (PI3K) to promote the RAF/mitogen or extracellular signal-regulated kinases (MEK/ERK) pathway, PI3K/AKT/mammalian target of rapamycin (mTOR) pathway and RalGDS (Ral guanine nucleotide dissociation stimulator) pathway (McCormick et al., J. Mol. Med. (Berl)., 2016, 94(3):253-8; Rodriguez-Viciana et al., Cancer Cell. 2005, 7(3):205-6). These pathways affect diverse cellular processes such as proliferation, survival, metabolism, motility, angiogenesis, immunity and growth (Young et al., Adv. Cancer Res., 2009, 102:1-17; Rodriguez-Viciana et al., Cancer Cell. 2005, 7(3):205-6).

Cancer-associated mutations in Ras family proteins suppress their intrinsic and GAP-induced GTPase activity leading to an increased population of GTP-bound/active mutant Ras family proteins (McCormick et al., Expert Opin. Ther. Targets., 2015, 19(4):451-4; Hunter et al., Mol. Cancer Res., 2015, 13(9):1325-35). This in turn leads to persistent activation of effector pathways (e.g. RAF/MEK/ERK, PI3K/AKT/mTOR, RalGDS pathways) downstream of Ras family proteins. KRAS mutations (e.g. amino acids G12, G13, Q61, A146) are found in a variety of human cancers including lung cancer, colorectal cancer and pancreatic cancer (Cox et al., Nat. Rev. Drug Discov., 2014, 13(11):828-51). Mutations in HRAS (e.g. amino acids G12, G13, Q61) and NRAS (e.g. amino acids G12, G13, Q61, A146) are also found in a variety of human cancer types however typically at a lower frequency compared to KRAS mutations (Cox et al., Nat. Rev. Drug Discov., 2014, 13(11):828-51). Alterations (e.g. mutation, over-expression, gene amplification) in Ras family proteins have also been described as a resistance mechanism against cancer drugs such as the EGFR antibodies cetuximab and panitumumab (Leto et al., J. Mol. Med. (Berl). 2014 July; 92(7):709-22) and the EGFR tyrosine kinase inhibitor osimertinib/AZD9291 (Ortiz-Cuaran et al., Clin. Cancer Res., 2016, 22(19):4837-47; Eberlein et al., Cancer Res., 2015, 75(12):2489-500).

Son of Sevenless 1 (SOS1) is a human homologue of the originally identified Drosophila protein Son of Sevenless (Pierre et al., Biochem. Pharmacol., 2011, 82(9):1049-56; Chardin et al., Cytogenet. Cell. Genet., 1994, 66(1):68-9). The SOS1 protein consists of 1333 amino acids (150 kDa). SOS1 is a multi-domain protein with two tandem N-terminal histone domains (HD) followed by the Dbl homology domain (DH), a Pleckstrin homology domain (PH), a helical linker (HL), RAS exchanger motif (REM), CDC25 homology domain and a C-terminal proline rich domain (PR). SOS1 has two binding sites for Ras family proteins; a catalytic site that binds GDP-bound Ras family proteins to promote guanine nucleotide exchange and an allosteric site that binds GTP-bound Ras family proteins which causes a further increase in the catalytic GEF function of SOS1 (Freedman et al., Proc. Natl. Acad. Sci. USA., 2006, 103(45):16692-7; Pierre et al., Biochem. Pharmacol., 2011, 82(9):1049-56). Published data indicate a critical involvement of SOS1 in mutant KRAS activation and oncogenic signaling in cancer (Jeng et al., Nat. Commun., 2012, 3:1168). Depleting SOS1 levels decreased the proliferation rate and survival of tumor cells carrying a KRAS mutation whereas no effect was observed in KRAS wild type cell lines. The effect of loss of SOS1 could not be rescued by introduction of a catalytic site mutated SOS1, demonstrating the essential role of SOS1 GEF activity in KRAS mutant cancer cells.

SOS1 is critically involved in the activation of Ras family protein signaling in cancer via mechanisms other than mutations in Ras family proteins. SOS1 interacts with the adaptor protein Grb2 and the resulting SOS1/Grb2 complex binds to activated/phosphorylated Receptor Tyrosine Kinases (e.g. EGFR, ErbB2, ErbB3, ErbB4, PDGFR-A/B, FGFR1/2/3, IGF1R, INSR, ALK, ROS, TrkA, TrkB, TrkC, RET, c-MET, VEGFR1/2/3, AXL) (Pierre et al., Biochem. Pharmacol., 2011, 82(9):1049-56). SOS1 is also recruited to other phosphorylated cell surface receptors such as the T cell Receptor (TCR), B cell Receptor (BCR) and monocyte colony-stimulating factor receptor (Salojin et al., J. Biol. Chem. 2000, 275(8):5966-75). This localization of SOS1 to the plasma membrane, proximal to Ras family proteins, enables SOS1 to promote Ras family protein activation. SOS1-activation of Ras family proteins can also be mediated by the interaction of SOS1/Grb2 with the BCR-ABL oncoprotein commonly found in chronic myelogenous leukemia (Kardinal et al., 2001, Blood, 98:1773-81; Sini et al., Nat. Cell Biol., 2004, 6(3):268-74).

Furthermore, alterations in SOS1 have been implicated in cancer. SOS1 mutations are found in embryonal rhabdomyosarcomas, sertoli cell testis tumors, granular cell tumors of the skin (Denayer et al., Genes Chromosomes Cancer, 2010, 49(3):242-52) and lung adenocarcinoma (Cancer Genome Atlas Research Network., Nature. 2014, 511(7511):543-50). Meanwhile over-expression of SOS1 has been described in bladder cancer (Watanabe et al., IUBMB Life., 2000, 49(4):317-20) and prostate cancer (Timofeeva et al., Int. J. Oncol., 2009, 35(4):751-60). In addition to cancer, hereditary SOS1 mutations are implicated in the pathogenesis of RASopathies like e.g. Noonan syndrome (NS), cardio-facio-cutaneous syndrome (CFC) and hereditary gingival fibromatosis type 1 (Pierre et al., Biochem. Pharmacol., 2011, 82(9):1049-56).

SOS1 is also a GEF for the activation of the GTPases RAC1 (Ras-related C3 botulinum toxin substrate 1) (Innocenti et al., J. Cell Biol., 2002, 156(1):125-36). RAC1, like RAS-family proteins, is implicated in the pathogenesis of a variety of human cancers and other diseases (Bid et al., Mol. Cancer Ther. 2013, 12(10):1925-34).

Son of Sevenless 2 (SOS2), a homolog of SOS1 in mammalian cells, also acts as a GEF for the activation of Ras family proteins (Pierre et al., Biochem. Pharmacol., 2011, 82(9):1049-56; Buday et al., Biochim. Biophys. Acta., 2008, 1786(2):178-87). Published data from mouse knockout models suggests a redundant role for SOS1 and SOS2 in homeostasis in the adult mouse. Whilst germline knockout of SOS1 in mice results in lethality during mid-embryonic gestation (Qian et al., EMBO J., 2000, 19(4):642-54), systemic conditional SOS1 knockout adult mice are viable (Baltanás et al., Mol. Cell. Biol., 2013, 33(22):4562-78). SOS2 gene targeting did not result in any overt phenotype in mice (Esteban et al., Mol. Cell. Biol., 2000, 20(17):6410-3). In contrast, double SOS1 and SOS2 knockout leads to rapid lethality in adult mice (Baltanás et al., Mol. Cell. Biol., 2013, 33(22):4562-78). These published data suggest that selective targeting of individual SOS isoforms (e.g. selective SOS1 targeting) may be adequately tolerated to achieve a therapeutic index between SOS1/Ras family protein driven cancers (or other SOS1/Ras family protein pathologies) and normal cells and tissues.

Selective pharmacological inhibition of the binding of the catalytic site of SOS1 to Ras family proteins is expected to prevent SOS1-mediated activation of Ras family proteins to the GTP-bound form. Such SOS1 inhibitors are expected to consequently inhibit signaling in cells downstream of Ras family proteins (e.g. ERK phosphorylation). In cancer cells associated with dependence on Ras family proteins (e.g. KRAS mutant cancer cell lines), SOS1 inhibitors are expected to deliver anti-cancer efficacy (e.g. inhibition of proliferation, survival, metastasis etc.). High potency towards inhibition of SOS1:Ras family protein binding (nanomolar level IC₅₀ values) and ERK phosphorylation in cells (nanomolar level IC₅₀ values) are desirable characteristics for a SOS1 inhibitor.

Several covalent KRAS G12C inhibitors, such as AMG 510 and MRTX849, are currently in clinical development. Data from KRAS G12C inhibitor clinical trials have shown so far the highest response rate for patients with NSCLC, whereas patients with CRC demonstrated a lower response rate. Preclinically, CDX and PDX models initially often respond to KRAS G12C inhibitor monotherapy treatment followed by relapse and outgrowth of tumors. Combination therapy of a KRAS G12C inhibitor may therefore lead to enhanced anti-tumor efficacy and durability of tumor response and may address or delay adaptive resistance mechanisms.

The efficacy of therapeutic agents can be improved by using combination therapies (in particular in oncology) with other compounds and/or improving the dosage schedule. Even if the concept of combining several therapeutic agents has already been suggested, and although various combination therapies are under investigation and in clinical trials, there is still a need for new and efficient therapeutic concepts and specific combinations for the treatment of cancer diseases, e.g. solid tumors, which show advantages over standard therapies, such as for example better treatment outcome, beneficial effects, superior efficacy, enhanced durability of tumor response and/or improved tolerability, such as e.g. reduced side effects of the combined treatment. Specifically, there is a need for additional treatment options for patients with cancers like, e.g., pancreatic cancer, lung cancer (e.g. NSCLC), colorectal cancer or cholangiocarcinoma.

It is thus an object of the present invention to provide combination treatments/methods of combination treatment providing certain advantages compared to treatments/methods of treatment currently used and/or known in the prior art. These advantages may include in vivo efficacy (e.g. improved clinical response, extend of the response, increase of the rate of response, duration of response, disease stabilization rate, duration of stabilization, time to disease progression, progression free survival (PFS) and/or overall survival (OS), later occurence of resistance and the like), safe and well tolerated administration and reduced frequency and severity of adverse events.

In this context, the inventors of the present invention, surprisingly, discovered that the use of specific inhibitors of the interaction between SOS1 and Ras family proteins (referred to herein as “SOS1 inhibitor”) in combination with specific KRAS G12C inhibitors have the potential to improve clinical outcome compared to the use of either the SOS1 inhibitor or the KRAS G12C inhibitor alone.

It is believed that treatment with a SOS1 inhibitor represses KRAS activation and shifts the equilibrium from the active GTP-KRAS form towards the inactive GDP-KRAS form. Consequently, SOS1 inhibitors have the potential to sensitize KRAS G12C mutant tumors to covalent KRAS G12C inhibitors that are only binding to the GDP-KRAS form. Furthermore, combination treatment of a SOS1 inhibitor and a KRAS G12C inhibitor may lead to a synergistic anti-proliferative effect, enhanced MAPK pathway modulation and apoptosis induction as well as blockade of adaptive feedback relief, i.e. enhanced efficacy in combination compared to both mono-therapies and the observed response may be more durable and well tolerated.

Thus, the invention relates to methods for the treatment and/or prevention of oncological and/or hyperproliferative diseases, in particular cancer, as described herein, comprising the combined administration of a SOS1 inhibitor and a KRAS G12C inhibitor, each as described herein, as well as to medical uses, to uses, to pharmaceutical compositions or combinations and kits comprising such therapeutic agents.

Further, the invention relates to anti-cancer therapies comprising using a SOS1 inhibitor and a KRAS G12C inhibitor, each as described herein, in combination.

For the treatment of diseases of oncological nature, a large number of anticancer agents (including target-specific and non-target-specific anticancer agents) have already been suggested, which can be used as monotherapy or as combination therapy involving more than one agent (e.g. dual or triple combination therapy) and/or which may be combined with radiotherapy (e.g. irradiation treatment), radio-immunotherapy and/or surgery.

It is a purpose of the present invention to provide combination therapies with the therapeutic agents described herein for treating or controlling various malignancies (e.g. based on cooperative, complementary, interactive or improving effects of the active components involved in combination).

### DETAILED DESCRIPTION OF THE INVENTION

(Medical) Uses—Methods of Treatment—Combinations—Compositions—Kits

Thus, in one aspect the invention relates to a method of treating and/or preventing an oncological and/or hyperproliferative disease, in particular cancer, as described herein, comprising administering to a patient a therapeutically effective amount of a SOS1 inhibitor and a therapeutically effective amount of a KRAS G12C inhibitor, each as described herein.

Such a combined treatment may be given as a non-fixed (e.g. free) combination of the substances or in the form of a fixed combination, including kit-of-parts.

In another aspect the invention relates to a combination of a SOS1 inhibitor and a KRAS G12C inhibitor, each as described herein, particularly for use in a method of treating and/or preventing an oncological and/or hyperproliferative disease, in particular cancer, as described herein, said method comprising administering to a patient a therapeutically effective amount of the combination.

In another aspect the invention relates to a SOS1 inhibitor as described herein for use in a method of treating and/or preventing an oncological and/or hyperproliferative disease, in particular cancer, as described herein, said method comprising administering the SOS1 inhibitor in combination with a KRAS G12C inhibitor as described herein to a patient.

In another aspect the invention relates to a KRAS G12C inhibitor as described herein for use in a method of treating and/or preventing an oncological and/or hyperproliferative disease, in particular cancer, as described herein, said method comprising administering the KRAS G12C inhibitor in combination with a SOS1 inhibitor as described herein to a patient.

In another aspect the invention relates to a kit comprising
a first pharmaceutical composition or dosage form comprising a SOS1 inhibitor as described herein, and, optionally, one or more pharmaceutically acceptable carriers, excipients and/or vehicles, and
a second pharmaceutical composition or dosage form comprising a KRAS G12C inhibitor as described herein, and, optionally, one or more pharmaceutically acceptable carriers, excipients and/or vehicles.

In another aspect the invention relates to the aforementioned kits further comprising
a package insert comprising printed instructions for simultaneous, concurrent, sequential, successive, alternate or separate use in the treatment and/or prevention of an oncological and/or hyperproliferative disease, in particular cancer, as described herein, in a patient.

In another aspect the invention relates to the aforementioned kits for use in a method of treating and/or preventing an oncological and/or hyperproliferative disease, in particular cancer, as described herein.

In another aspect the invention relates to a pharmaceutical composition comprising
a SOS1 inhibitor as described herein,
a KRAS G12C inhibitor as described herein, and
optionally, one or more pharmaceutically acceptable carriers, excipients and/or vehicles.

In another aspect the invention relates to the use of a SOS1 inhibitor as described herein for the manufacture of a medicament for use in a method of treating and/or preventing an oncological and/or hyperproliferative disease, in particular cancer, as described herein, wherein the medicament is to be used in combination with a KRAS G12C inhibitor as described herein.

In another aspect the invention relates to the use of a KRAS G12C inhibitor as described herein for the manufacture of a medicament for use in a method of treating and/or preventing an oncological and/or hyperproliferative disease, in particular cancer, as described herein, wherein the medicament is to be used in combination with a SOS1 inhibitor as described herein.

In another aspect the invention relates to the use of a SOS1 inhibitor and a KRAS G12C inhibitor, each as described herein, for the manufacture of a medicament for use in a method of treating and/or preventing an oncological and/or hyperproliferative disease, in particular cancer, as described herein.

In another aspect the invention relates to a combination, a pharmaceutical composition, a medicament or a kit according to the invention, each as described herein, comprising, consisting or consisting essentially of a SOS1 inhibitor and a KRAS G12C inhibitor, each as described herein, for use in a method of treating and/or preventing an oncological and/or hyperproliferative disease, in particular cancer, as described herein.

SOS1 Inhibitor

Preferably, the SOS1 inhibitor within this invention and all its embodiments (including methods of treatment, (medical) uses, combinations, compositions etc.) is selected from the group consisting of example compounds I-1 to I-179 or salts thereof as disclosed in PCT application no. PCT/EP2018/086197 (WO 2019/122129), the disclosure (which includes the respective synthesis and properties) being incorporated herein by reference in its entirety [A0].

More preferably, the SOS1 inhibitor within this invention and all its embodiments (including methods of treatment, (medical) uses, combinations, compositions etc.) is selected from the group consisting of the following specific SOS1 inhibitors or pharmaceutically acceptable salts thereof (table A) [A1]

TABLE A

| | |
|---|---|
| I-1 | 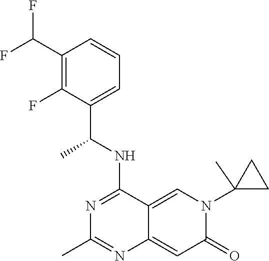 [SMILES: Cc1nc(N[C@H](C)c2cccc(C(F)F)c2F)c2cn(C3(C)CC3)c(=O)cc2n1] |
| I-2 | 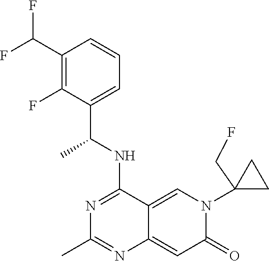 [SMILES: Cc1nc(N[C@H](C)c2cccc(C(F)F)c2F)c2cn(C3(CF)CC3)c(=O)cc2n1] |
| I-3 | 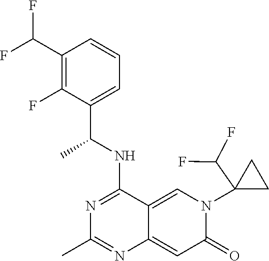 [SMILES: Cc1nc(N[C@H](C)c2cccc(C(F)F)c2F)c2cn(C3(C(F)F)CC3)c(=O)cc2n1] |
| I-21 | 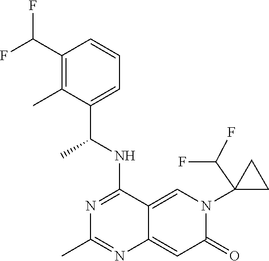 [SMILES: Cc1nc(N[C@H](C)c2cccc(C(F)F)c2C)c2cn(C3(C(F)F)CC3)c(=O)cc2n1] |
| I-52 | 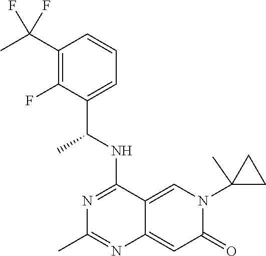 [SMILES: Cc1nc(N[C@H](C)c2cccc(C(C)(F)F)c2F)c2cn(C3(C)CC3)c(=O)cc2n1] |
| I-53 | 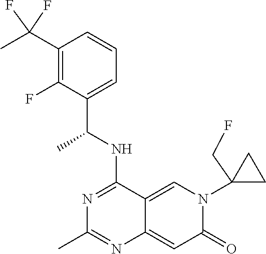 [SMILES: Cc1nc(N[C@H](C)c2cccc(C(C)(F)F)c2F)c2cn(C3(CF)CC3)c(=O)cc2n1] |
| I-54 | 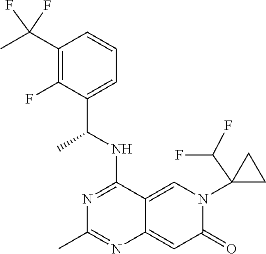 [SMILES: Cc1nc(N[C@H](C)c2cccc(C(C)(F)F)c2F)c2cn(C3(C(F)F)CC3)c(=O)cc2n1] |
| I-55 | 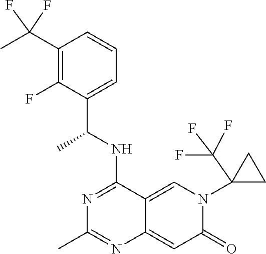 [SMILES: Cc1nc(N[C@H](C)c2cccc(C(C)(F)F)c2F)c2cn(C3(C(F)(F)F)CC3)c(=O)cc2n1] |
| I-58 | 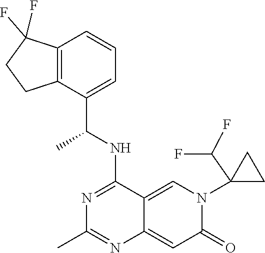 [SMILES: Cc1nc(N[C@H](C)c2cccc3c2CCC3(F)F)c2cn(C3(C(F)F)CC3)c(=O)cc2n1] |
| I-77 | 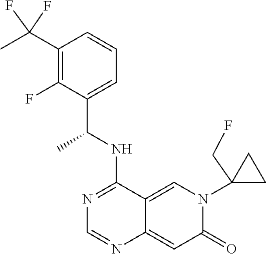 [SMILES: C[C@@H](Nc1ncnc2cc(=O)n(C3(CF)CC3)cc12)c1cccc(C(C)(F)F)c1F] |
| I-82 | 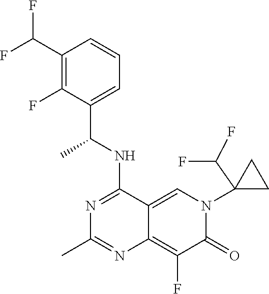 [SMILES: Cc1nc(N[C@H](C)c2cccc(C(F)F)c2F)c2cn(C3(C(F)F)CC3)c(=O)c(F)c2n1] |
| I-97 | 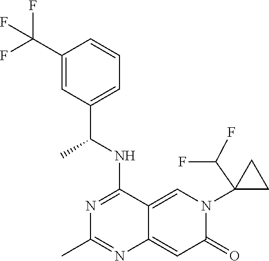 [SMILES: Cc1nc(N[C@H](C)c2cccc(C(F)(F)F)c2)c2cn(C3(C(F)F)CC3)c(=O)cc2n1] |
| I-98 | 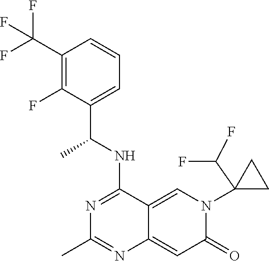 [SMILES: Cc1nc(N[C@H](C)c2cccc(C(F)(F)F)c2F)c2cn(C3(C(F)F)CC3)c(=O)cc2n1] |
| I-99 | 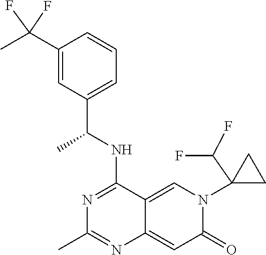 [SMILES: Cc1nc(N[C@H](C)c2cccc(C(C)(F)F)c2)c2cn(C3(C(F)F)CC3)c(=O)cc2n1] |
| I-102 | 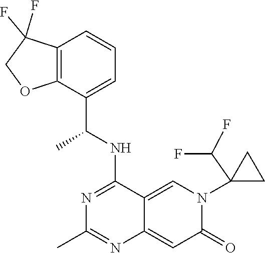 [SMILES: Cc1nc(N[C@H](C)c2cccc3c2OCC3(F)F)c2cn(C3(C(F)F)CC3)c(=O)cc2n1] |
| I-103 | 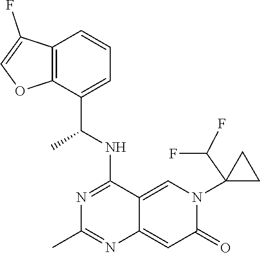 [SMILES: Cc1nc(N[C@H](C)c2cccc3c(F)coc23)c2cn(C3(C(F)F)CC3)c(=O)cc2n1] |

The term “SOS1 inhibitor” as used herein also includes the SOS1 inhibitors listed above in the form of a tautomer, of a pharmaceutically acceptable salt, of a hydrate or of a solvate (including a hydrate or solvate of a pharmaceutically acceptable salt). It also includes the SOS1 inhibitor in all its solid, preferably crystalline, forms and in all the crystalline forms of its pharmaceutically acceptable salts, hydrates and solvates (including hydrates and solvates of pharmaceutically acceptable salts).

In one embodiment the SOS1 inhibitor is compound I-1 in table A or a pharmaceutically acceptable salt thereof [A2].

In another embodiment the SOS1 inhibitor is compound I-2 in table A or a pharmaceutically acceptable salt thereof [A3].

In another embodiment the SOS1 inhibitor is compound I-3 in table A or a pharmaceutically acceptable salt thereof [A4].

In another embodiment the SOS1 inhibitor is compound I-21 in table A or a pharmaceutically acceptable salt thereof [A5].

In another embodiment the SOS1 inhibitor is compound I-52 in table A or a pharmaceutically acceptable salt thereof [A6].

In another embodiment the SOS1 inhibitor is compound I-53 in table A or a pharmaceutically acceptable salt thereof [A7].

In another embodiment the SOS1 inhibitor is compound I-54 in table A or a pharmaceutically acceptable salt thereof [A8].

In another embodiment the SOS1 inhibitor is compound I-55 in table A or a pharmaceutically acceptable salt thereof [A9].

In another embodiment the SOS1 inhibitor is compound I-58 in table A or a pharmaceutically acceptable salt thereof [A10].

In another embodiment the SOS1 inhibitor is compound I-77 in table A or a pharmaceutically acceptable salt thereof [A1 1].

In another embodiment the SOS1 inhibitor is compound I-82 in table A or a pharmaceutically acceptable salt thereof [A12].

In another embodiment the SOS1 inhibitor is compound I-97 in table A or a pharmaceutically acceptable salt thereof [A13].

In another embodiment the SOS1 inhibitor is compound I-98 in table A or a pharmaceutically acceptable salt thereof [A14].

In another embodiment the SOS1 inhibitor is compound I-99 in table A or a pharmaceutically acceptable salt thereof [A15].

In another embodiment the SOS1 inhibitor is compound I-102 in table A or a pharmaceutically acceptable salt thereof [A16].

In another embodiment the SOS1 inhibitor is compound I-103 in table A or a pharmaceutically acceptable salt thereof [A17].

All embodiments [A1] to [A17] are preferred embodiments of embodiment [A0] in respect of the nature of the SOS1 inhibitor.

KRAS G12C Inhibitor

Preferably, the KRAS G12C inhibitor within this invention and all its embodiments (including methods of treatment, (medical) uses, combinations, compositions etc.) is selected from the group consisting of the following specific KRAS G12C inhibitors or pharmaceutically acceptable salts thereof (table B) [B0]:

TABLE B

| | |
|---|---|
| B | 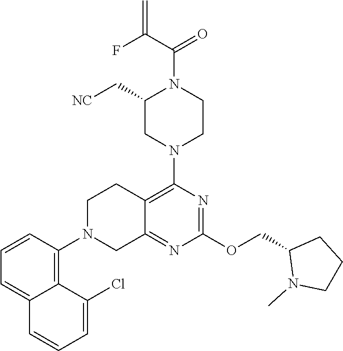 [SMILES: C=C(F)C(=O)N1CCN(c2nc(OC[C@@H]3CCCN3C)nc3c2CCN(c2cccc4cccc(Cl)c24)C3)C[C@@H]1CC#N] |
| C | 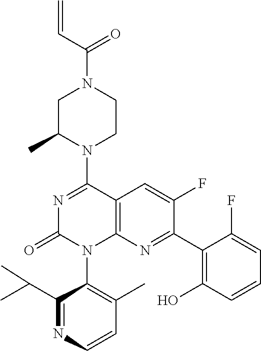 |

Compound B is also known under lab code MRTX849. PCT applications WO 2017/201161 and WO 2019/099524 (and patent family members thereof) describe general reaction schemes for preparing compound B and also provide detailed synthetic routes for preparation. The properties of compound B are also described in these applications and elsewhere.

Compound C is also known under lab code AMG 510 and the proposed INN sotorasib. PCT applications WO 2018/217651 and WO 2020/102730 (and patent family members thereof) describe general reaction schemes for preparing compound C and also provide detailed synthetic routes for preparation. The properties of compound C are also described in these applications and elsewhere.

The term “KRAS G12C inhibitor” as used herein also includes the KRAS G12C inhibitors listed above in the form of a tautomer, of a pharmaceutically acceptable salt, of a hydrate or of a solvate (including a hydrate or solvate of a pharmaceutically acceptable salt). It also includes the KRAS G12C inhibitor in all its solid, preferably crystalline, forms and in all the crystalline forms of its pharmaceutically acceptable salts, hydrates and solvates (including hydrates and solvates of pharmaceutically acceptable salts).

In one embodiment the KRAS G12C inhibitor is compound B in table B or a pharmaceutically acceptable salt thereof [B1].

In another embodiment the KRAS G12C inhibitor is compound C in table B or a pharmaceutically acceptable salt thereof [B2].

Embodiments [B1] and [B2] are preferred embodiments of embodiment [B0] in respect of the nature of the KRAS G12C inhibitor.

The combination of embodiments [A0] to [A17] (in respect of the nature of the SOS1 inhibitor) with embodiments [B0] to [B2] (in respect of the nature of the KRAS G12C inhibitor) results in specific dual combinations or groups of dual combinations which shall all be deemed to be specifically disclosed and to be embodiments of the invention and of all of its combinations, compositions, kits, methods, uses and compounds for use. Preferred combinations of embodiments [A] and [B] are listed in Table C:

TABLE C

| Combination # | SOS1 inhibitor | KRAS G12C inhibitor |
|---|---|---|
| 1 | I-1 | B |
| 2 | I-2 | B |
| 3 | I-3 | B |
| 4 | I-21 | B |
| 5 | I-52 | B |
| 6 | I-53 | B |
| 7 | I-54 | B |
| 8 | I-55 | B |
| 9 | I-58 | B |
| 10 | I-77 | B |
| 11 | I-82 | B |
| 12 | I-97 | B |
| 13 | I-98 | B |
| 14 | I-99 | B |
| 15 | I-102 | B |
| 16 | I-103 | B |
| 17 | I-1 | C |
| 18 | I-2 | C |
| 19 | I-3 | C |
| 20 | I-21 | C |
| 21 | I-52 | C |
| 22 | I-53 | C |
| 23 | I-54 | C |
| 24 | I-55 | C |
| 25 | I-58 | C |
| 26 | I-77 | C |
| 27 | I-82 | C |
| 28 | I-97 | C |
| 29 | I-98 | C |
| 30 | I-99 | C |
| 31 | I-102 | C |
| 32 | I-103 | C |

The term “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, and commensurate with a reasonable benefit/risk ratio.

As used herein “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

By a “therapeutically effective amount” for the purposes of this invention is meant a quantity of substance that is capable of obviating symptoms of illness or of preventing or alleviating these symptoms, or which prolong the survival of a treated patient.

To be used in therapy, the SOS1 inhibitor and the KRAS G12C inhibitor, separately or jointly, are included into pharmaceutical compositions appropriate to facilitate administration to animals or humans.

Typical pharmaceutical compositions for administering the SOS1 inhibitor and the KRAS G12C inhibitor, separately or jointly, include for example tablets, capsules, suppositories, solutions, e.g. solutions for injection (s.c., i.v., i.m.) and infusion, elixirs, emulsions or dispersible powders. The content of the pharmaceutically active compound(s) may be in the range from 0.1 to 90 wt.-%, preferably 40 to 60 wt.-% of the composition as a whole, e.g. in amounts which are sufficient to achieve the desired dosage range. The single dosages may, if necessary, be given several times a day to deliver the desired total daily dose.

Typical tablets may be obtained, for example, by mixing the active substance(s), optionally in combination, with known excipients, for example inert diluents such as calcium carbonate, calcium phosphate, cellulose or lactose, disintegrants such as corn starch or alginic acid or crospovidon, binders such as starch or gelatine, lubricants such as magnesium stearate or talc and/or agents for delaying release, such as carboxymethyl cellulose, cellulose acetate phthalate, or polyvinyl acetate. The tablets may be prepared by usual processes, such as e.g. by direct compression or roller compaction. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substance(s) may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing the active substance(s) may for example be prepared by mixing the active substance(s) with inert carriers such as lactose or sorbitol and packing them into gelatine capsules.

Typical suppositories may be made for example by mixing the active substance(s) with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

Thus, the SOS1 inhibitor and KRAS G12C inhibitor of this invention and all its embodiments can be optionally administered in the form of a pharmaceutical composition as herein described.

The SOS1 inhibitor and KRAS G12C inhibitor of this invention and all its embodiments is administered by the usual methods, preferably by oral or parenteral route, most preferably by oral route. For oral administration the tablets may contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for oral use for SOS1 inhibitors, in particular for SOS1 inhibitors in table A, is from 1 mg to 2000 mg per dose (e.g. 10 mg to 1000 mg per dose; in a more preferred embodiment from 200 mg to 600 mg per dose; most preferred is from 400 mg to 500 mg per dose). In one embodiment a single dose comprises 50 mg of the SOS1 inhibitor. In another embodiment a single dose comprises 100 mg of the SOS1 inhibitor. In another embodiment a single dose comprises 200 mg of the SOS1 inhibitor. In another embodiment a single dose comprises 400 mg of the SOS1 inhibitor. In another embodiment a single dose comprises 800 mg of the SOS1 inhibitor. In another embodiment a single dose comprises 1600 mg of the SOS1 inhibitor. In another embodiment a single dose comprises 2000 mg of the SOS1 inhibitor. All amounts given refer to the free base of the SOS1 inhibitor and may be proportionally higher if a pharmaceutically acceptable salt or other solid form is used.

In one embodiment the SOS1 inhibitor, in particular a SOS1 inhibitor in table A, is dosed once daily (q.d.).

The dosage for oral use for KRAS G12C inhibitor compound B is from 10 mg to 1000 mg per dose (e.g. 400 mg to 800 mg per dose). In one embodiment a single dose comprises 600 mg of compound B. All amounts given refer to the free base of compound B and may be proportionally higher if a pharmaceutically acceptable salt or other solid form is used.

In one embodiment the KRAS G12C inhibitor compound B is dosed once daily (q.d.). In a preferred embodiment compound B is dosed twice daily (b.i.d.).

The dosage for oral use for KRAS G12C inhibitor compound C is from 100 mg to 1500 mg per dose (e.g. 300 mg to 1000 mg per dose). In one embodiment a single dose comprises 360 mg of compound C. In another embodiment a single dose comprises 720 mg of the compound C. In another embodiment a single dose comprises 960 mg of compound C. All amounts given refer to the free base of compound C and may be proportionally higher if a pharmaceutically acceptable salt or other solid form is used.

In one embodiment the KRAS G12C inhibitor compound C is dosed once daily (q.d.).

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

Combination Therapy

Within this invention it is to be understood that the combinations, compositions, kits, methods, uses or compounds for use according to this invention may envisage the simultaneous, concurrent, sequential, successive, alternate or separate administration of the active ingredients or components. It will be appreciated that the SOS1 inhibitor and the KRAS G12C inhibitor, both as described herein, can be administered formulated either dependently or independently, such as e.g. the SOS1 inhibitor and the KRAS G12C inhibitor may be administered either as part of the same pharmaceutical composition/dosage form or, preferably, in separate pharmaceutical compositions/dosage forms.

In this context, “combination” or “combined” within the meaning of this invention includes, without being limited, a product that results from the mixing or combining of more than one active ingredient and includes both fixed and non-fixed (e.g. free) combinations (including kits) and uses, such as e.g. the simultaneous, concurrent, sequential, successive, alternate or separate use of the components or ingredients. The term “fixed combination” means that the active ingredients are both administered to a patient simultaneously in the form of a single entity or dosage. The term “non-fixed combination” means that the active ingredients are both administered to a patient as separate entities either simultaneously, concurrently or sequentially with no specific time limits, wherein such administration provides therapeutically effective levels of the two compounds in the body of the patient.

The administration of the SOS1 inhibitor and the KRAS G12C inhibitor may take place by co-administering the active components or ingredients, such as e.g. by administering them simultaneously or concurrently in one single or in two or more separate formulations or dosage forms. Alternatively, the administration of the SOS1 inhibitor and the KRAS G12C inhibitor may take place by administering the active components or ingredients sequentially or in alternation, such as e.g. in two or more separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

The elements of the combinations of this invention may be administered (whether dependently or independently) by methods customary to the skilled person, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Accordingly, in one aspect of the invention the invention provides a method of treating and/or preventing an oncological and/or hyperproliferative disease, in particular cancer, as described herein, comprising administering to a patient a therapeutically effective amount of SOS1 inhibitor and a therapeutically effective amount of a KRAS G12C inhibitor, each as described herein, wherein the SOS1 inhibitor is administered simultaneously, concurrently, sequentially, successively, alternately or separately with the KRAS G12C inhibitor.

In another aspect the invention provides a SOS1 inhibitor as described herein for use in a method of treating and/or preventing an oncological and/or hyperproliferative disease, in particular cancer, as described herein, said method comprising administering the SOS1 inhibitor in combination with a KRAS G12C inhibitor as described herein, wherein the SOS1 inhibitor is administered simultaneously, concurrently, sequentially, successively, alternately or separately with the KRAS G12C inhibitor.

In another aspect the invention provides a KRAS G12C inhibitor as described herein for use in a method of treating and/or preventing an oncological and/or hyperproliferative disease, in particular cancer, as described herein, said method comprising administering the KRAS

G12C inhibitor in combination with a SOS1 inhibitor as described herein, wherein the KRAS G12C inhibitor is administered simultaneously, concurrently, sequentially, successively, alternately or separately with the SOS1 inhibitor.

In another aspect the invention provides the use of a SOS1 inhibitor as described herein for the manufacture of a medicament for use in a method of treating and/or preventing an oncological and/or hyperproliferative disease, in particular cancer, as described herein, wherein the medicament is to be used in combination with a KRAS G12C inhibitor as described herein, and wherein the medicament is to be administered simultaneously, concurrently, sequentially, successively, alternately or separately with the KRAS G12C inhibitor.

In another aspect the invention provides a kit comprising
a first pharmaceutical composition or dosage form comprising a SOS1 inhibitor and, optionally, one or more pharmaceutically acceptable carriers, excipients and/or vehicles, and
a second pharmaceutical composition or dosage form comprising a KRAS G12C inhibitor, and, optionally, one or more pharmaceutically acceptable carriers, excipients and/or vehicles, for use in a method of treating and/or preventing an oncological and/or hyperproliferative disease, in particular cancer, as described herein, wherein the first pharmaceutical composition or dosage form is to be administered simultaneously, concurrently, sequentially, successively, alternately or separately with the second pharmaceutical composition or dosage form.

In a further embodiment of the invention the components (i.e. the combination partners) of the combinations, kits, uses, methods and compounds for use according to the invention (including all embodiments) are administered simultaneously.

In a further embodiment of the invention the components (i.e. the combination partners) of the combinations, kits, uses, methods and compounds for use according to the invention (including all embodiments) are administered concurrently.

In a further embodiment of the invention the components (i.e. the combination partners) of the combinations, kits, uses, methods and compounds for use according to the invention (including all embodiments) are administered sequentially.

In a further embodiment of the invention the components (i.e. the combination partners) of the combinations, kits, uses, methods and compounds for use according to the invention (including all embodiments) are administered successively.

In a further embodiment of the invention the components (i.e. the combination partners) of the combinations, kits, uses, methods and compounds for use according to the invention (including all embodiments) are administered alternately.

In a further embodiment of the invention the components (i.e. the combination partners) of the combinations, kits, uses, methods and compounds for use according to the invention (including all embodiments) are administered separately.

The combinations of this invention may be administered at therapeutically effective single or divided daily doses. The active components of the combination may be administered in such doses which are therapeutically effective in monotherapy, or in such doses which are lower or higher than the doses used in monotherapy, but when combined result in a desired (joint) therapeutically effective amount.

The combinations, compositions, kits, (medical) uses, methods and compounds for use according to the present invention (including all embodiments) including a SOS1 inhibitor and a KRAS G12C inhibitor, each as described herein, may optionally include one or more additional therapeutic agent(s).

Oncological and/or Hyperproliferative Diseases—Cancers

The combinations, compositions, kits, uses, methods and compounds for use according to the present invention (including all embodiments) are useful for the treatment and/or prevention of oncological and/or hyperproliferative diseases.

In certain embodiments, the oncological and/or hyperproliferative disease is cancer.

In certain embodiments, the oncological and/or hyperproliferative disease is cancer with tumor cells harboring a G12C mutant KRAS gene.

In certain embodiments, the oncological and/or hyperproliferative disease is a cancer selected from the group consisting of pancreatic cancer, lung cancer, colorectal cancer, cholangiocarcinoma, appendiceal cancer, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas, wherein optionally but preferably the cancer is characterized by tumor cells harboring a G12C mutant KRAS gene.

In certain embodiments, the oncological and/or hyperproliferative disease is a cancer selected from the group consisting of pancreatic cancer, lung cancer (preferably non-small cell lung cancer (NSCLC), colorectal cancer and cholangiocarcinoma, wherein optionally but preferably the cancer is characterized by tumor cells harboring a G12C mutant KRAS gene.

In certain embodiments, the oncological and/or hyperproliferative disease is non-small cell lung cancer (NSCLC) (including for example locally advanced or metastatic NSCLC (stage IIIB/IV), NSCLC adenocarcinoma, NSCLC with squamous histology, NSCLC with non-squamous histology), wherein optionally but preferably the non-small cell lung cancer is characterized by tumor cells harboring a G12C mutant KRAS gene.

In certain embodiments, the oncological and/or hyperproliferative disease is non-small cell lung cancer (NSCLC), in particular NSCLC adenocarcinoma, wherein optionally but preferably the non-small cell lung cancer is characterized by tumor cells harboring a G12C mutant KRAS gene.

In certain embodiments, the oncological and/or hyperproliferative disease is colorectal cancer, wherein optionally but preferably the colorectal cancer is characterized by tumor cells harboring a G12C mutant KRAS gene.

In certain embodiments, the oncological and/or hyperproliferative disease is pancreatic cancer, wherein optionally but preferably the pancreatic cancer is characterized by tumor cells harboring a G12C mutant KRAS gene.

In certain embodiments, the oncological and/or hyperproliferative disease is cholangiocarcinoma, wherein optionally but preferably the cholangiocarcinoma is characterized by tumor cells harboring a G12C mutant KRAS gene.

In particularly preferred embodiments, the oncological and/or hyperproliferative disease is selected from the group consisting of:
lung adenocarcinoma harboring a KRAS G12C mutation;
colorectal adenocarcinoma harboring a KRAS G12C mutation; and
pancreatic adenocarcinoma harboring a KRAS G12C mutation.

Additionally, the following cancers, tumors and other oncological and/or hyperproliferative diseases may be treated with combinations, compositions, kits, uses, methods and compounds for use according to the present invention (including all embodiments), wherein optionally but preferably the cancer, tumor or other oncological and/or hyperproliferative disease is characterized by tumor cells harboring a G12C mutant KRAS gene:
cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas,
gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VlPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP);

All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:

Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;

Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas;

The therapeutic applicability of the combination therapy according to this invention may include first line, second line, third line or further lines of treatment of patients. The cancer may be metastatic, recurrent, relapsed, resistant or refractory to one or more anti-cancer treatments. Thus, the patients may be treatment naïve, or may have received one or more previous anti-cancer therapies, which have not completely cured the disease.

Patients with relapse and/or with resistance to one or more anti-cancer agents (e.g. the single components of the combination, or standard chemotherapeutics) are also amenable for combined treatment according to this invention, e.g. for second or third line treatment cycles (optionally in further combination with one or more other anti-cancer agents), e.g. as add-on combination or as replacement treatment.

Accordingly, some of the disclosed combination therapies of this invention are effective at treating subjects whose cancer has relapsed, or whose cancer has become drug resistant or multi-drug resistant, or whose cancer has failed one, two or more lines of mono- or combination therapy with one or more anti-cancer agents (e.g. the single components of the combination, or standard chemotherapeutics).

A cancer which initially responded to an anti-cancer drug can relapse and become resistant to the anti-cancer drug when the anti-cancer drug is no longer effective in treating the subject with the cancer, e.g. despite the administration of increased dosages of the anti-cancer drug. Cancers that have developed resistance to two or more anti-cancer drugs are said to be multi-drug resistant.

Accordingly, in some methods of combination treatment of this invention, treatment with a combination according to this invention administered secondly or thirdly is begun if the patient has resistance or develops resistance to one or more agents administered initially or previously. The patient may receive only a single course of treatment with each agent or multiple courses with one, two or more agents.

In certain instances, combination therapy according to this invention may hence include initial or add-on combination, replacement or maintenance treatment.

1. (canceled)

2. (canceled)

3. (canceled)

4. (canceled)

5. (canceled)

6. A pharmaceutical composition comprising:
a SOS1 inhibitor is selected from the group consisting of: 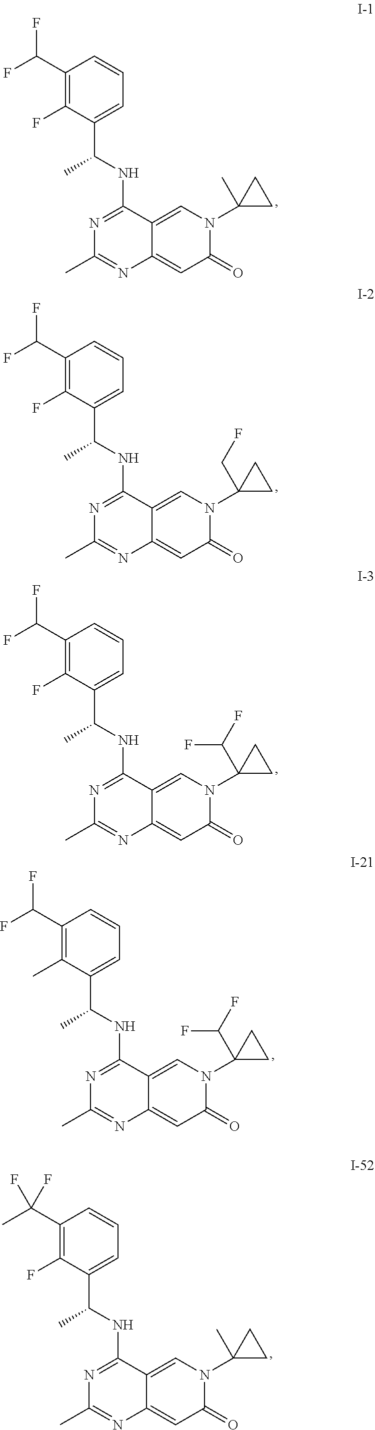 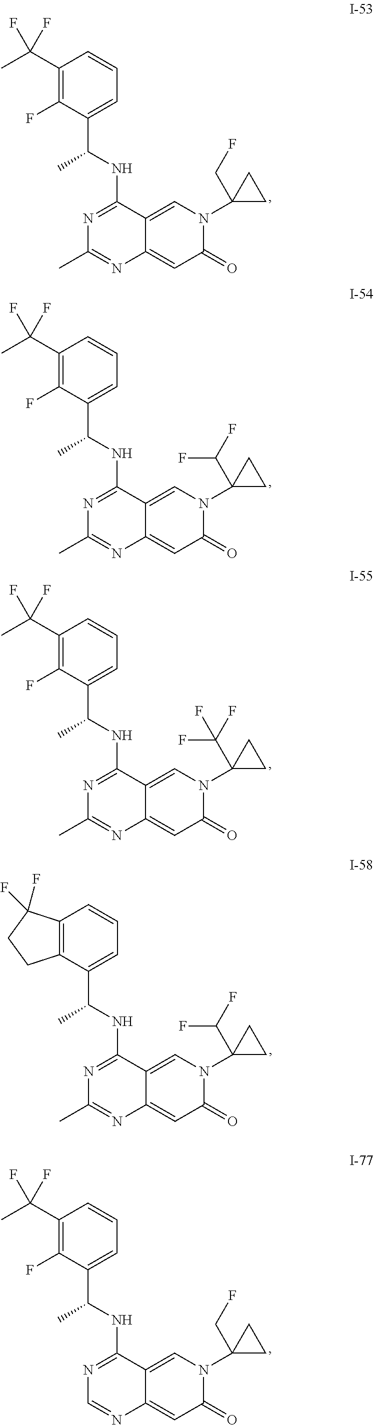 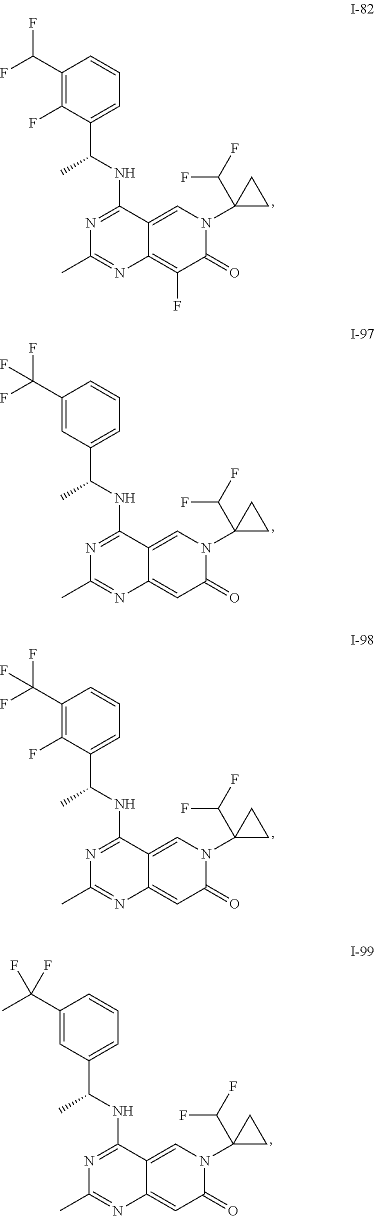 [SMILES: Cc1nc(N[C@H](C)c2cccc(C(F)F)c2F)c2cn(C3(C(F)F)CC3)c(=O)c(F)c2n1 ; Cc1nc(N[C@H](C)c2cccc(C(F)(F)F)c2)c2cn(C3(C(F)F)CC3)c(=O)cc2n1 ; Cc1nc(N[C@H](C)c2cccc(C(F)(F)F)c2F)c2cn(C3(C(F)F)CC3)c(=O)cc2n1 ; Cc1nc(N[C@H](C)c2cccc(C(C)(F)F)c2)c2cn(C3(C(F)F)CC3)c(=O)cc2n1] 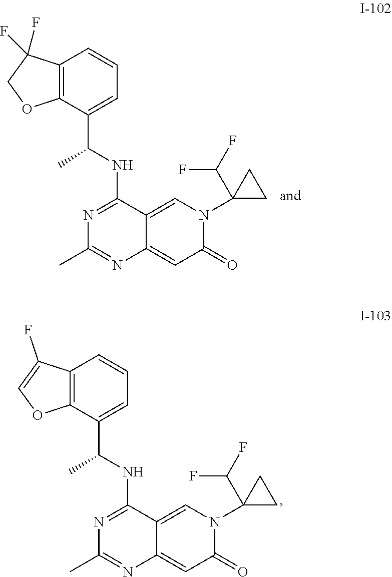 [SMILES: Cc1nc(N[C@H](C)c2cccc3c2OCC3(F)F)c2cn(C3(C(F)F)CC3)c(=O)cc2n1 ; Cc1nc(N[C@H](C)c2cccc3c(F)coc23)c2cn(C3(C(F)F)CC3)c(=O)cc2n1]
or a pharmaceutically acceptable salt thereof,
a KRAS G12C inhibitor is selected from the group consisting of: 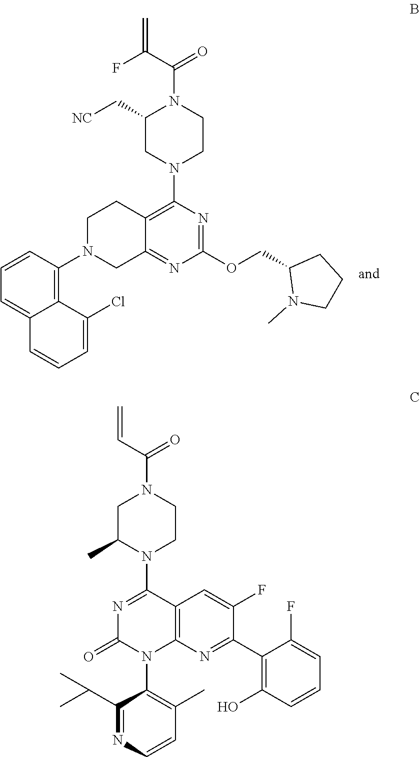
or a pharmaceutically acceptable salt thereof,
and, optionally, one or more pharmaceutically acceptable carriers, excipients and/or vehicles.

7. A method of treating and/or preventing an oncological and/or hyperproliferative disease, comprising administering to a patient a therapeutically effective amount of the pharmaceutical composition according to claim 6.

8. A kit comprising:
a first pharmaceutical composition or dosage form comprising a SOS1 inhibitor as defined in claim 6 and, optionally, one or more pharmaceutically acceptable carriers, excipients and/or vehicles,
a second pharmaceutical composition or dosage form comprising a KRAS G12C inhibitor as defined in claim 6 and, optionally, one or more pharmaceutically acceptable carriers, excipients and/or vehicles.

9. A method of treating and/or preventing an oncological and/or hyperproliferative disease, in particular cancer comprising administering to a patient therapeutically effective amounts of the first and second pharmaceutical compositions in the kit according to claim 8.

10. The method according to claim 9, wherein the first pharmaceutical composition is to be administered simultaneously, concurrently, sequentially, successively, alternately or separately with the second pharmaceutical composition.

11. The kit according to claim 8 further comprising
a package insert comprising printed instructions for simultaneous, concurrent, sequential, successive, alternate or separate use of the first and second pharmaceutical compositions in the treatment and/or prevention of an oncological and/or hyperproliferative disease in a patient.

12. A method of treating and/or preventing an oncological and/or hyperproliferative disease, comprising administering to a patient a therapeutically effective amount of a SOS1 inhibitor and a therapeutically effective amount of a KRAS G12C inhibitor, wherein the SOS1 inhibitor and the KRAS G12C inhibitor are as defined in claim 46.

13. The method according to claim 12, wherein the SOS1 inhibitor is administered simultaneously, concurrently, sequentially, successively, alternately or separately with the KRAS G12C inhibitor.

14. (canceled)

15. (canceled)

16. The method according to claim 7, wherein the oncological and/or hyperproliferative disease to be treated and/or prevented is a cancer selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

17. The method according to claim 16, wherein the oncological and/or hyperproliferative disease to be treated and/or prevented is selected from lung cancer, non-small cell lung cancer (NSCLC), NSCLC adenocarcinoma, colorectal cancer, pancreatic cancer and cholangiocarcinoma.

18. The method according to claim 16, wherein the cancer to be treated and/or prevented harbors a KRAS G12C mutation.

19. The method according to claim 9, wherein the oncological and/or hyperproliferative disease to be treated and/or prevented is a cancer selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

20. The method according to claim 19, wherein the oncological and/or hyperproliferative disease to be treated and/or prevented is selected from lung cancer, non-small cell lung cancer (NSCLC), NSCLC adenocarcinoma, colorectal cancer, pancreatic cancer and cholangiocarcinoma.

21. The method according to claim 19, wherein the cancer to be treated and/or prevented harbors a KRAS G12C mutation.

22. The method according to claim 12, wherein the oncological and/or hyperproliferative disease to be treated and/or prevented is a cancer selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

23. The method according to claim 22, wherein the oncological and/or hyperproliferative disease to be treated and/or prevented is selected from lung cancer, non-small cell lung cancer (NSCLC), NSCLC adenocarcinoma, colorectal cancer, pancreatic cancer and cholangiocarcinoma.

24. The method according to claim 22, wherein the cancer to be treated and/or prevented harbors a KRAS G12C mutation.
